(19) United States
(12) Patent Application Publication
(10) Pub. No.: US 2022/0383978 A1
(43) Pub. Date: Dec. 1, 2022

(54) ACCELERATED METHOD FOR GENERATING TARGET ELITE INBREDS WITH SPECIFIC AND DESIGNED TRAIT MODIFICATION

(71) Applicant: Monsanto Technology LLC, St. Louis, MO (US)

(72) Inventors: Humberto I. Gutierrez, Wildwood, MO (US); Uche Godfrey Okeke, St. Louis, MO (US); Nathaniel J. Page, Madison, WI (US); Curtis W. Scherder, Ames, IA (US); Slobodan Trifunovic, Ankeny, IA (US); Lei Zhu, Ballwin, MO (US)

(21) Appl. No.: 17/707,600

(22) Filed: Mar. 29, 2022

Related U.S. Application Data
(60) Provisional application No. 63/172,462, filed on Apr. 8, 2021.

(51) Int. Cl. G16B 20/40; G16B 25/10; A01H 1/04

(52) U.S. Cl. CPC G16B 20/40; G16B 25/10; A01H 1/045

(57) ABSTRACT

The present disclosure provides a method of generating a new trait converted elite cultivar through a method of breeding. For instance, the method involves the use of parent plants, which are respectively the traited variant of the parents of the non-traited elite cultivar and estimating a minimum population size necessary to generate a progeny plant comprising the desired trait and sharing a sufficiently high identity by descent with the non-traited elite cultivar to ensure replication and equivalency of general performance. The present method may be used to generate an elite cultivar in fewer generations, thereby accelerating new line production, and reducing costs. The present method may also be used to generate non-traited variants of traited lines.

### REFERENCE TO RELATED APPLICATION

This application claims the benefit of U.S. Provisional Application No. 63/172,462, filed Apr. 8, 2021, which is herein incorporated by reference in its entirety.

### FIELD OF THE INVENTION

The present disclosure relates to the field of agricultural biotechnology, and more specifically to crop breeding.

### BACKGROUND OF THE INVENTION

Breeders are continually developing new cultivars through various plant genetic improvement programs. These programs commonly rely on both forward breeding methods and backcross based trait introgression methods. These methods can be time consuming and inefficient. As breeders look to accelerate crop variety development, it is critical to develop improved methods for developing new cultivars that increase efficiency and facilitate a faster generation of new cultivars.

### SUMMARY OF THE INVENTION

Disclosed is a method of creating a trait-carrying version of a target line directly via forward breeding methods instead of introgression. In one embodiment, the method includes providing a first parent and a second parent. In another embodiment, the first parent and second parent are parents from which the target line was originally derived. In yet another embodiment, at least one of the parents carries a trait-of-interest. In still yet another embodiment, the parents are inbreds. The method may involve two parental inbreds both of which are trait-of-interest carrying variants of the parents from which the target line was originally derived. In another embodiment, the method also includes generating a population of traited progeny by crossing the first and second parents. In a further embodiment, the method further includes determining a minimum population size of the traited progeny population based at least on the genetic information associated with each of the parents, and the target line, such that regeneration of the trait-variant of the target line can be achieved with sufficiently high probability. In yet another embodiment, the population of traited progeny generated is equal to or greater than the size of the determined minimum population size. The method may also include selecting, from the traited population, at least one line based on the genetic similarity between the newly generated line and the target line to become a traited replica and variant of the target line.

In a further embodiment, determining or estimating the minimum population size of the traited progeny population comprises generating a set of virtual genomes by simulating recombinations of the genomes of the first parent and the second parent; estimating a similarity between each member of the set of virtual genomes and a genome of the target line; comparing each of the estimated similarities to a similarity threshold; determining a proportion of the set of virtual genomes whose estimated similarities to the genome of the target line is equal to or exceeds the similarity threshold; and estimating, based on the proportion, a probability of a recombination between the first parent and the second parent of generating a line whose similarity to the target line is equal to or exceeds the similarity threshold. In another embodiment, the method includes using an identity-by-descent method, for instance, a haploid-based identity-by-descent method, to estimate the similarity between each member of the set of virtual genomes and the genome of the target line.

In another embodiment, the method comprises crossing the traited target line with the target line. The target line may be an inbred line, for instance, a corn inbred line, or a rapeseed inbred line. In still another embodiment, the trait of interest comprises at least one agronomic trait of interest, for instance an agronomic trait of interest associated with any combination of herbicide tolerance, insect control, increased plant pathogen resistance, enhanced oil composition, increased water use efficiency, increased yield, increased drought resistance, increased seed quality, improved nutritional quality, increased nitrogen use efficiency, or tolerance to nitrogen stress.

In one embodiment, the method also includes regenerating a trait-carrying target line with the original pair of parents with only one of which replaced with a trait-carrying variant. In a further embodiment, the method also includes using at least one line from the newly generated trait-carry population, not as a direct replica of the target line, but as a trait donor line for the introgression of the trait into the target line via a classic trait integration method.

Also disclosed is a system of creating a traited-carrying version of a target line directly via forward breeding methods instead of introgression. In one embodiment, the system includes a breeding pipeline of a target environmental region. In another embodiment, the system also includes a first parent and second parent that are parents from which the target line was originally derived. In yet another embodiment, at least one of the parents carries a trait-of-interest. In still yet another embodiment, the parents are inbreds. The method may involve two parental inbreds both of which are trait-of-interest carrying variants of the parents from which the target line was originally derived. In one embodiment, the system additionally includes a computing device in communication with a data structure and configured to determining a minimum population size of a population of traited progeny by crossing the first and second parents based at least on the genetic information associated with each of the parents, and the target line, such that regeneration of the trait-variant of the target line can be achieved with sufficiently high probability. Methods of estimating or determining a minimum population size are provided herein. In another embodiment, the system includes a means of generating a traited progeny population by crossing the first parent with the second parent. In yet another embodiment, the population of traited progeny generated is equal to or greater than the size of the determined minimum population size. In a further embodiment, the system further includes a means of selecting, from the traited population, at least one line based on the genetic similarity between the newly generated line and the target line to become a traited replica and variant of the target line. In yet a further embodiment, the system also includes planting a plant derived from at least one traited variant of the target line in a growing space and directing it into the breeding pipeline.

In one embodiment, the system also includes regenerating a trait-carrying target line with the original pair of parents with only one of which replaced with a trait-carrying variant. The system may also include using at least one line from the newly generated trait-carry population, not as a direct replica of the target line, but as a trait donor line for the introgression of the trait into the target line via a classic trait integration method.

Also disclosed is a method of generating a genetically modified version of a target line. In one embodiment, the method includes providing a first parent and a second parent. In another embodiment, the first parent and second parent are parents from which the target line was originally derived. In yet another embodiment, at least one of the parents carries a trait-of-interest. In still yet another embodiment, the parents are inbreds. In a further embodiment, the method involves estimating a minimum population size of the genetically modified progeny population based at least on genetic information associated with the first and second parents, and the target line. Methods of estimating or determining a minimum population size are provided herein. In yet a further embodiment, the method involves generating a genetically modified progeny population by crossing the first parent with the second parent. In another embodiment, the size of the genetically modified progeny population is equal to or greater than the estimated minimum genetically modified progeny population size. The method may comprise selecting, from the genetically modified progeny population, at least one genetically modified target line based on a genetic similarity between the genetically modified target line and the target line. In a further embodiment, the method comprises crossing the genetically modified target line with the target line.

### BRIEF DESCRIPTION OF THE DRAWINGS

FIG. 1—Diagrammatical representation of development of new elite inbred lines through bi-parental crosses (P1 and P2), including development of early lines followed by multiple years of testing, screening, and selection of desired new elite inbred lines (i.e. Line1).

FIG. 2—Diagrammatical representation of an exemplary method to create parental lines comprising a trait of interest (P1-Trait and P2-Trait) using traditional backcross-based trait introgression methods.

FIG. 3—Diagrammatical representation of method for establishing an identity by descent threshold by developing a haplotype-based identity by descent method to infer the amount of genomic sharing between a base germplasm and a version of the base germplasm in which a desired trait of interest has been introgressed. Field-based traited-testing are carried out to establish the threshold of the percentage recipient parental plant identity-by-descent recovery as the quality metric and predictor for base germplasm performance reproducibility.

FIG. 4—Diagrammatical representation of in-silico simulation of the recombination of the genomes of the parents (P1-T and P2-T) of the elite germplasm and estimation of identity by descent (IBD) between the original elite germplasm (Line1) and each virtual recombinant.

FIG. 5—Diagrammatical representation of the method for estimating the probability of success of reaching at least the previously determined identity by descent (IBD) threshold (classifier) between the original elite line and each of the virtual recombinants.

FIG. 6—Diagrammatical representation of determining the minimum population size (N) of double haploids (DH) necessary to obtain a trait converted plant having the desired identity by descent to the original elite line, using the probability of success (p) of reaching at least the previously determined identity by descent and the percent level of reliability (i.e. 95%).

FIG. 7—Diagrammatical representation of different breeding processes for trait introgression, where the genetic distance between the original parent plants is too large, requiring a population size that exceeds feasibility. In such a situation, a backcross to the elite germplasm (Line 1) is simulated and the resulting progeny (BC1) are used for determination of population size (shown in panel a). Alternatively, a cross between full-sibling plants may be simulated and the resulting progeny used for determination of population size (shown in panel b).

FIG. 8—Diagrammatical representation of different breeding processes for trait introgression. Line Breeding Process represents the traditional trait introgression line breeding process. The Reverse Selection (F1DH) variant is designed to achieve direct recovery of the targeted genome straight from the F1 recombinants.

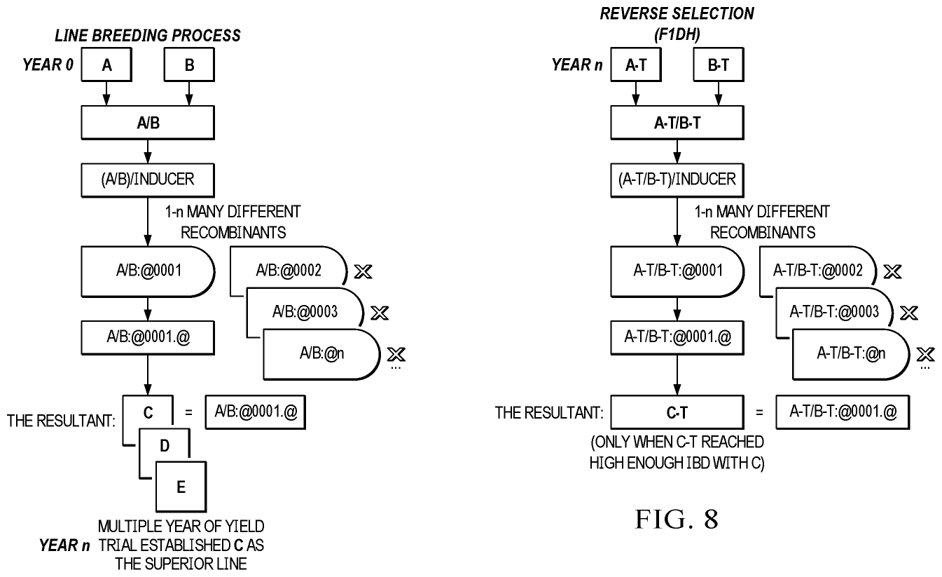

FIG. 9—Diagrammatical representation of different breeding processes for trait introgression. The Reverse Selection (F1DH-BC) variant is designed to achieve a slightly lower portion of target genome recovery that F1DH variant, and by design requires an additional backcross(es) to achieve the same level of target genome recovery as in F1DH. The Reverse Selection (F1-Donor-BC) design essentially drops the double-haploid steps and uses the genetic segregating F1 progenies as donors and achieves comparable level of target genome recovery.

FIG. 10—Diagrammatical representation of an example of trait integration using a traited parent line.

FIG. 11—Diagrammatical representation of another example of generating traited variant of a target line using a traited parent line.

FIG. 12—Diagrammatical representation of an example of generating non-traited lines using reverse selection.

### DETAILED DESCRIPTION

The development of new cultivars through the combination of desirable traits with an elite genome traditionally requires the creation of new elite lines and trait integration into such elite lines, requiring multiple generations of crossing and backcrossing. The final goal of such repeated backcrossing is to achieve a plant containing both the desired trait from a donor parent plant and a high recovery of an elite recurrent parent plant's genome to ensure performance replication of the elite recurrent parent plant. This final goal often requires an ultra-high identity by descent and can therefore be costly and require multiple years to complete, even with the assistance of multiple cycles per year and winter nurseries, marker assisted breeding methods, or protected culture operations.

The present disclosure provides a novel method of developing cultivars through a new plant breeding method to reduce the time, and thus cost, required to develop new trait integrated elite lines. Specifically, the presently disclosed method involves the creation of at least one parent plant of an elite line comprising the trait or traits of interest and estimating the minimum population size necessary to reliably obtain a progeny plant comprising a desired genetic similarity to the non-traited elite line. Thus, the method is a re-production of the non-traited elite, but now with a specific trait as the additional modification. Whereas the traditional breeding methods rely on selection, this method is the opposite in that a breeding target line is pre-defined and then breeding population is created to produce such a target as close as possible. The presently disclosed method therefore is described as a “reverse selection” to reduce the required time and costs to develop new trait integrated elite lines of the corresponding targeted non-traited elite lines.

### A. Advantages of Reverse Selection

For transgenic crops and markets with high GMO penetration and dominance, the modern seed and trait industry must deliver two goals simultaneously to remain competitive: good overall genome and good specific genome segments.

The former is commonly achieved via a forward breeding approach, which involves crossing elite germplasms to generate new recombinant genomes and progeny, thus representing novel patterns of reshuffling of existing haplotypes. Multi-stage, genotypic or phenotypic-based screening and selection may then be applied to identify and validate new inbred plants from the resulting progeny that are reliably better than the inbred parental plants over a wide range of agronomics and yield performance. These new elite lines are often a very tiny fraction of the size of the starting population, diminishing the return on a very costly breeding and product-by-trial process.

The latter is commonly achieved via backcross-based trait integration approach, which involves crossing a donor parent plant to a recipient or recurrent parent plant. The donor parent plant is typically an inbred carrying a trait of interest, and recipient parent plant is a plant representing the desired over-all genomic composition. The trait of interest may be a biotech trait but may also be a strong and well-definite QTL sufficiently small enough to be introgressed through breeding efficiently. As the intention is to introduce the trait of interest from the donor parent plant, and preserve the recipient parent plant genome to the fullest extent possible, multi-generations of backcrossing and genotype-base selection may be applied, to achieve the adequate recovery of the recipient parent genome. Thus, the recipient parent genome serves as a blue print and is the target of the backcross and marker-based selection process. The level of adequate recovery may be established with two components: a genomic similarity algorithm, and equivalency test, which is a phenotypic and field test establishing the similarity threshold for key performance reproducibility between the base (non-traited) germplasm and the converted version of the germplasm, comprising the trait. Thus, in contrast to forward breeding, trait integration is essentially a product-by-design process aiming for faithful recovery of the recipient parent genome or performance.

To achieve a balance between the two arms of this parallel system is a fundamental challenge in the seed and trait industry. It is undesirable to start the trait integration process too early and prematurely in the main breeding process, due to fact that the new elite inbred pool under evaluation may be too large and the inbred plants are still considered crude instead of refined and valuable enough to serve as appropriate recipient parent plants. Starting trait integration too late is equally undesirable. At such a later-stage, the inbred plants may be much more refined design targets, and the confidence on their superior performance may be much higher. However, multiple backcross generations may be required to achieve high recipient plant genome recovery, causing delays for market launch. On the other hand, avoiding such delays by simply reducing the number of backcross generations, or reducing the targeted level of recurrent parent recovery in the final product is counter-productive. Doing so increases the probability of converted lines failing to reproduce performance of the base inbred germplasm and places the investment of multiple years of testing and screening at risk.

Additionally, the ever-increasing complexity of trait stacking poses a further challenge to the traditional backcross-based trait integration methods. Multiple independently segregating loci each introduce small amount of linkage drag, and the accumulation of such drag reduces the effectiveness of the recipient parent genome recovery per backcross generation. A 6-trait conversion may therefore potentially require more backcross generations to complete than a 2-trait conversion, even when the donor parent and recipient parent genetic distances are the same. The additional Mendelian segregation associates with multiple segregating traits increase the size, and the investment, required during the selfing stage for the recovery of multi-loci homozygous trait-positives. As a comparison, the probability of recovering homozygotes from a 2-trait hemi-heterozygote is 0.0625, while this probability reduces to 2.44×10⁻⁴ for a 6-trait heterozygote independently segregating.

The breeding method disclosed herein addresses some of the challenges in the art, summarized above. For instance, the presently disclosed method may reduce the time and cost associated with generating new trait converted elite cultivars through the use of traited parent lines to produce donor F1 plants. The classic trait integration process typically involves at least three backcross cycles, requiring two years, to reach the necessary genomic and performance recovery of the recipient parent. The presently disclosed method provides a novel trait donor strategy to shorten trait integration process.

In further embodiments, using haplotype analysis and genetics simulations, the presently disclosed method determines for each suitable inbred origin, the required population size, and if any, additional backcross generations required to reach the conversion quality comparable to that from a traditional backcross-base trait integration. In some embodiments, the presently disclosed method may be suitable for multiple-segregating trait conversion, where a backcross approach may be less effective than a single trait conversion.

### B. Reverse Selection Methods

The presently disclosed method facilitates the production of new elite lines comprising a trait or traits of interest, and in certain embodiments, reduces the cost and time associated with development of such lines.

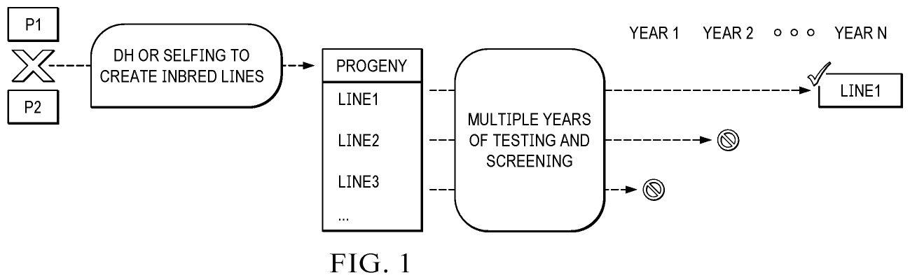

Initially, an elite germplasm is identified or created for use as a recipient plant for introgression of a desired trait (FIG. 1). Development of such elite germplasm may be accomplished by any method known by those of skill in the art. For instance, one such non-limiting method may involve bi-parental development crosses between parental inbred plants followed by a multi-year breeding program involving phenotype and/or genotype-based screening and selection assays to identify the resulting elite line candidates. The parental inbred plants may be traited or non-traited. For instance, the parental inbred plants may comprise one or more genetic loci conferring a trait of interest. Additional methods and specific breeding steps and screening or selection assays would be readily understood to one of skill in the art, and any method known in the art to develop a new elite germplasm may be used in accordance with the presently disclosed method.

As sibling lines typically have a high similarity to each other, it would be desirable to use sibling lines as trait donor lines. However, it is difficult to find a sibling line suitable as trait donor line for classical trait introgression methods. This conversely is caused by no other than the long duration it takes to conduct trait integration itself: if it takes 3 years to conduct trait integration, at any given time the available donors are unlikely to be sibling lines, but instead older lines that were generated a few years prior. To address this, the presently disclosed method involves, in one embodiment, the recreation of a pool of traited F1 progeny, from which a line of the highest similarity is to be identified as donor, by crossing traited version of the original parental elite lines.

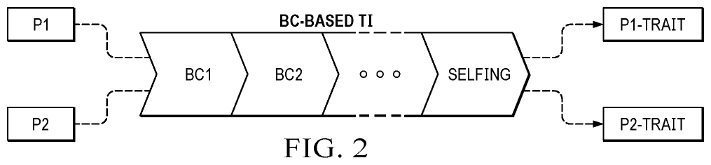

To generate traited F1 progeny, traited versions of one or both parental lines of the elite line may be generated (FIG. 2). For instance, versions of the parental plants of the elite line that comprise one or more genetic loci conferring one or more traits of interest that the parental lines did not previously comprise may be developed. In some embodiments, these traited lines may be developed using any method known in the art to introduce or introgress a genetic locus conferring a trait, including, but not limited to breeding, for instance, backcrossing, or genetic manipulation, for instance, transformation or genome editing.

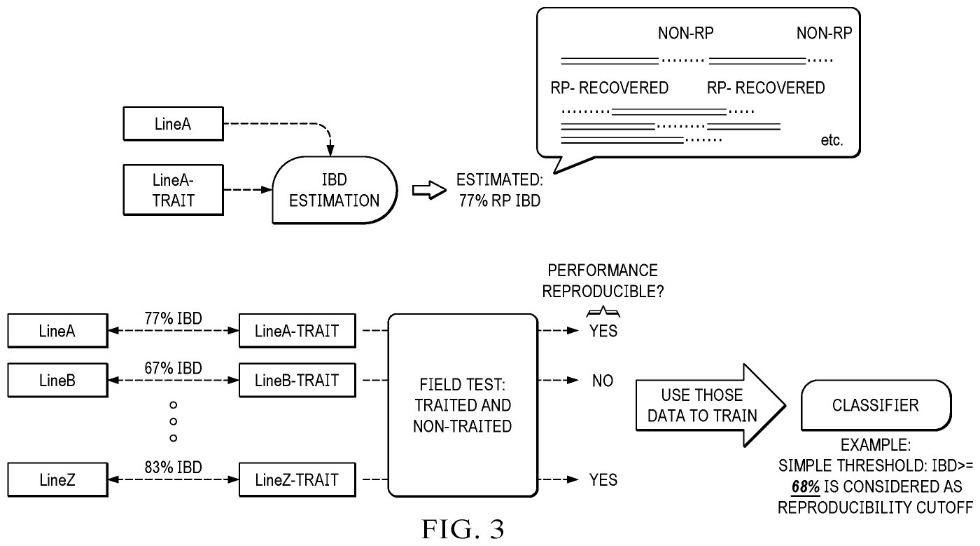

In a further embodiment, the presently disclosed method involves establishing a method to measure genetic similarity, for instance by developing a haplotype-based identity by descent method, to infer the amount of genomic sharing between a base germplasm and a version of the base germplasm in which a desired trait of interest has been introgressed. Field-based traited-testing are carried out to establish the threshold of the percentage recipient parental plant identity by descent recovery for base germplasm performance reproducibility. This is performed using existing backcross converted lines and their matching base germplasm as training set. The information obtained from the already established backcross converted lines and matching base germplasm is then used to calculate desired identity by descent threshold representative of the necessary identity by descent required to obtain an acceptable level of performance reproducibility (FIG. 3).

A genetic simulation may then be conducted to generate virtual recombinations of genomes of the original parent plants of the elite germplasm developed earlier. Genetic simulation of chromosome recombination may, for example, be based on a map function mathematical model, giving for instance theoretical expectation of the cross-over probability or the length of recombined chromosome segment. Such genetic simulations may be produced using any means known in the art, including any of the well-known mathematical models in the art. In certain embodiments, the genetic simulation may be performed using any of the publicly available computer programs suitable for generating such simulated recombinations. Multiple publicly available software platforms are available to perform the in-silico recombinations, including but not limited to QGENE, MORGAN, simuPOP, genomeSIMLA, SysGenSIM, and others.

The presently disclosed method therefore provides, in some embodiments, a computing device in communication with a data structure and configured to perform genetic recombination simulations. For instance, the computing device may be configured to generate genetic recombination simulations between the genomes of the parental plants of the elite germplasm.

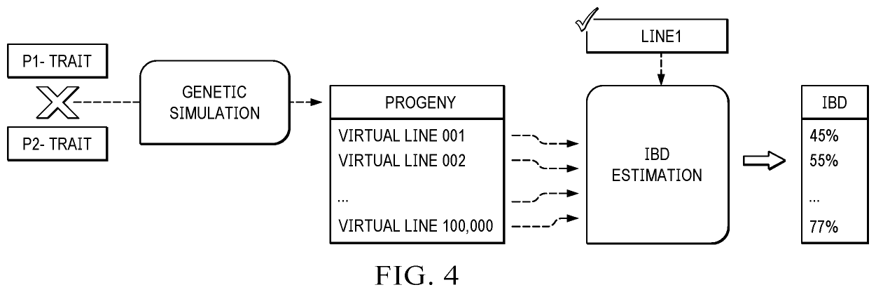

In certain embodiments, 10⁶˜10⁷ virtual recombinations are simulated. The virtual recombinant may be used as an in-silico representation of F1-derived from double haploid inbreds. The original parents of the elite germplasm are used for the simulation. The strong similarity, although not identical, between the original parents and the traited version of the parents allow their use in the simulation as a representation of the traited parents, which in one embodiment may be used in the later breeding steps. The haplotype-based identity by descent method described above is then performed for each of the virtual recombinants simulated, and the proportion of haplotype sharing between each virtual recombinant and the elite germplasm developed earlier is estimated (FIG. 4).

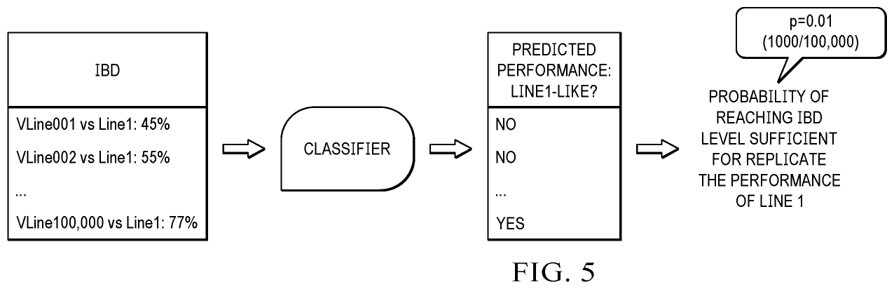

In another embodiment, the presently disclosed method next involves estimating the probability of success of reaching at least the previously determined threshold of identity by descent between the original elite germplasm and each of the virtual recombinants. This is accomplished by comparing the empirical distribution of identity by descent estimates obtained from the haplotype-based identity by descent method performed for each of the virtual recombinants simulated, to the previously established reproducibility threshold and estimating the empirical probability of reaching an identity by descent value equal-or-greater than the established threshold (FIG. 5).

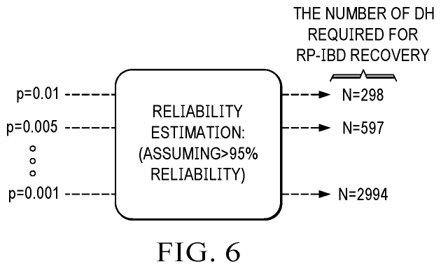

Based on the estimated probability of success of reaching at least the identity by descent threshold (p), and desired reliability level, an adequate population size is determined to reliably exceed the previously established reproducibility threshold (FIG. 6). For example, when the probability of exceeding the identity by descent target, such as 98.76% similarity, is p=0.001, a pool of 3000 of F1-derived double haploid inbreds is required to achieve ˜95% reliability (1−(1−0.001)³⁰⁰⁰). In some embodiments, any statistically relevant level of reliability may be used to determine the population size. For instance, the level of reliability may be 70%, 75%, 80%, 85%, 90%, 91%, 92%, 93%, 94%, 95%, 96%, 97%, 98%, or 99%. In certain embodiments, the population size is determined for F1-derived double haploids.

In other embodiments, where there is a large genetic distance between the inbred parents of the elite germplasm, the estimated population size exceeds practical feasibility, as a high parental distance may reduce probability of success of reaching at least the identity by descent threshold (p). For instance, in the case of p=0.0005, a population size of 6000 would be required to reach the 95% reliability level. In cases where the estimated population size exceeds practical feasibility, the genetic simulation performed to simulate virtual recombinants the may be modified to determine the population size required to reach an equivalent of 1 backcross, or alternatively, one full-sib cross, lower than the established identity by descent threshold, instead of exceeding the established threshold directly from within the F1-derived double haploid pool. The precise relationship between the number of backcross and the identity by descent metric may also be established by the genetic simulation performed to simulate virtual recombinants.

In further embodiments, once the required population size is determined, a pool of that number of trait-carrying inbreds is generated from which a final inbred line is identified. In some embodiments, the final inbred line may be created through direct double haploid recovery, by doubling a haploid copy of the genome of the F1 progeny plants created from the cross of the parent plants, where at least one of the parent plants has had a trait of interest introduced. In other embodiments, where the genetic distance between the original parent plants is large enough that the estimated population size exceeds practical feasibility, the final inbred line may be created by doubling a haploid copy of the genome of the F1 progeny plants created from the cross of the parent plants, where at least one of the parent plants has had a trait of interest introduced, followed by at least one generation of backcross to the elite germplasm used as the recipient plant. In some embodiments, when a backcross is required before generation of the final inbred, additional Mendelian segregation of the trait-of-interest may occur and additional genotypic- or event-based screening may be performed to identify trait-positive backcross (F1BC1) progeny. In yet a further embodiment, where the genetic distance between the original parent plants is large enough that the estimated population size exceeds practical feasibility, the final inbred line may be created by doubling a haploid copy of the genome of the F1 progeny plants created from the cross of the parent plants, where at least one of the parent plants has had a trait of interest introduced, followed by at least on generation of crossing to a full-sibling plant.

In embodiments requiring either one or more backcross of full-sibling crosses generations, a further genetic simulation may be performed for these crosses, and final population size may be determined using this simulation, thus providing a more feasible population size.

The presently disclosed method additionally can be customized to suit desired new elite germplasm delivery timelines and budgetary targets. In certain embodiments, this can be achieved, for instance, by employing different breeding methods, such as replacing haploid doubling with selfing or single-seed-descent, or adjusting the design parameters to attain a target number of generations or backcross or sibling crosses to arrive at the final product.

### C. Sources of Genetic Loci or Traits for Introgression

Genetic loci conferring traits for introgression from a donor parent may come from any source known in the art. For instance, in certain non-limiting embodiments, such genetic loci may be simply native genes, inherited genes, quantitative trait loci (QTL) that control quantitative expression of complex traits; or transgenes inserted into a recipient host plant or donor plant by a method of genetic engineering technologies, such as transformation or site-specific modification. Alternatively, the genetic modification may be by alternative engineering techniques, such as mutation, cloning, tilling, or other methods known to the art.

Desirable qualitative or agronomic traits include resistance to plant pathogens or pests, for example resistance to one or more of a viral disease, a bacterial disease, a fungal disease, a nematode disease and an insect pest. They may also be traits for tolerance to an herbicide, for example, inhibitors of 5-enolpyruvylshikimate-3-phosphate synthase (EPSPS), such as glyphosate; synthetic auxins, such as dicamba and 2,4-D; glutamine synthetase inhibitors, such as glufosinate; and acetyl CoA carboxylase (ACCase) inhibitors, such as quizalofop and haloxyfop. Other, non-limiting, desirable traits may include traits altering oil content or composition; water use efficiency; yield; drought resistance; seed quality; nutritional quality; nitrogen use efficiency; or tolerance to nitrogen stress.

In certain embodiments, donor parent plants may be selected on the basis of desirable qualitative or agronomic traits. The donor parent plant may contain one or more desirable trait for introgression. In some embodiments, the donor parent plant and recipient parent plant may be of the same taxa, while in others the donor parent plant and recipient parent plant may be of different but related taxa. Similarly, the donor parent plant or recipient parent plant each be an elite plant or cultivar, or the donor parent plant or recipient parent plant may a non-elite plant. In certain embodiments, optimization of donor parent plant choice can be done using techniques known in the art, for instance similar to those in classic trait introgression.

### C. Selection and Detection of Traits for Introgression

Where the desired trait or trait of interest is a plant phenotype trait, selection for a desired trait may be by any of the ways known in the art, for example detecting or quantifying an expressed trait (selection criterion). In some cases, the trait of interest may be easily monitored by the presence or absence of a marker sequence known to be linked to the gene(s) controlling the trait of interest. This will be true in those cases where the trait has been introduced by a genetic modification to the donor parent. In other instances, the trait may be detected based on the phenotype. Any similar or other process for detecting the trait may therefore be used, as is known in the art.

In particular embodiments of the presently disclosed method, marker-assisted selection may be used to select backcross progeny, identify the trait of interest, or increase the efficiency any other step in the present method. Genetic markers that can be used in the practice of the presently disclosed method include, but are not limited to, restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), DNA Amplification Fingerprinting (DAF), Sequence Characterized Amplified Regions (SCARs), Arbitrary Primed Polymerase Chain Reaction (AP-PCR), isozymes, and other markers known to those skilled in the art.

In certain embodiments of the disclosure, polymorphic markers can be used to detect a desired trait. Polymorphic markers may also serve as useful tools for assaying plants for determining the genetic distance or degree of identity between lines or varieties. For instance, polymorphic markers can assist in determining the degree of identity by descent between lines or varieties used as donor plants or recipient plants, or between simulated recombinant lines and the original elite or recipient line.

Nucleic acid-based analyses for determining the presence or absence of the genetic polymorphism (i.e. for genotyping) can be used in the method of the present disclosure. A wide variety of genetic markers for the analysis of genetic polymorphisms are available and known to those of skill in the art. The analysis may be used to identify or select for desired traits, or in certain embodiments to identify the genetic distance, for instance the degree of identity by descent, between plants in a population or between simulated recombinant lines and the original elite or recipient line.

As used herein, nucleic acid analysis methods include, but are not limited to, genotype by sequencing, DNA fingerprinting, PCR-based detection methods (for example, TaqMan assays), microarray methods, mass spectrometry-based methods and/or nucleic acid sequencing methods. In certain embodiments, the genetic distance between plants within a population, such as the genetic distance between simulated recombinant lines and the original elite or recipient line, may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

One method of achieving such amplification employs the polymerase chain reaction (PCR) (Mullis et al. (1986) Cold Spring Harbor Symp. Quant. Biol. 51:263-273; European Patent 50,424; European Patent 84,796; European Patent 258,017; European Patent 237,362; European Patent 201,184; U.S. Pat. Nos. 4,683,202; 4,582,788; and 4,683,194), using primer pairs that are capable of hybridizing to the proximal sequences that define a polymorphism in its double-stranded form. Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entirety. However, the compositions and methods of the presently disclosed method can be used in conjunction with any polymorphism typing method to detect polymorphisms in genomic DNA samples. These genomic DNA samples used include but are not limited to, genomic DNA isolated directly from a plant, cloned genomic DNA, or amplified genomic DNA.

For instance, polymorphisms in DNA sequences can be detected by hybridization to locus-specific oligonucleotide (ASO) probes as disclosed in U.S. Pat. Nos. 5,468,613 and 5,217,863. U.S. Pat. No. 5,468,613 discloses locus specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods, for example as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. Typing of target sequences by microarray-based methods is described in U.S. Pat. Nos. 6,799,122; 6,913,879; and 6,996,476.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR, forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

In another embodiment, a locus interest, for instance conferring a trait interest, or the genome of plants useful in the presently disclosed method, can be directly sequenced using nucleic acid sequencing technologies. Methods for nucleic acid sequencing are known in the art and include technologies provided by 454 Life Sciences (Branford, Conn.), Agencourt Bioscience (Beverly, Mass.), Applied Biosystems (Foster City, Calif.), LI-COR Biosciences (Lincoln, Nebr.), NimbleGen Systems (Madison, Wis.), Illumina (San Diego, Calif.), and VisiGen Biotechnologies (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays.

### Definitions

For purpose of clarity in reading the following specification and appended claims, the following terms and expressions shall have the meanings provided, wherein:

As used herein a “trait,” “desired trait,” “trait of interest,” or “trait of agronomic interest,” refers to a phenotype conferred by a particular allele, gene, or grouping of genes at a locus or loci in the genome of a plant. In certain embodiments, a trait of the present disclosure may be a trait related to suitability for a crop end-use or may be a trait that provides a commercial value. A trait of the present disclosure may comprise, but limited to, herbicide tolerance, insect control, increased plant pathogen resistance, enhanced oil composition, enhanced oil content, increased water use efficiency, increased yield, increased drought resistance, increased seed quality, improved nutritional quality, increased nitrogen use efficiency, or tolerance to nitrogen stress.

As used herein, a “locus,” or “genetic locus” refers to fixed position on a genomic sequence. The term “loci” is the plural form of the term “locus.” A locus may refer to a nucleotide position at a reference point on a chromosome, such as a position from the end of the chromosome. A locus may comprise genetic material, including but not limited to a genetic marker, or a gene, such as a transgene, or a native gene.

As used herein, an “allele” refers to one or more alternative forms of a genomic sequence at a given locus on a chromosome. In a diploid cell or organism, the two alleles of a given gene occupy corresponding loci on a pair of homologous chromosomes. Two or more alleles constitutes a polymorphism. The polymorphic sites of any nucleic acid sequence can be determined by comparing the nucleic acid sequences at one or more loci.

As used herein, a “haplotype” refers to one or more DNA variations, or polymorphisms (such as single nucleotide polymorphisms (SNPs)), or a combination of alleles found on the same chromosome that tend to be inherited together.

As used herein, a “marker” refers to a detectable characteristic that can be used to discriminate between alleles or organisms. Examples of such characteristics include, but are not limited to, genetic markers.

As used herein, the term “genotype” refers to the specific allelic makeup of a plant.

As used herein, the term “phenotype” refers to the detectable characteristics of a cell or organism, which characteristics are the manifestation of gene expression and thus influenced by genotype.

As used herein, “identity by descent” refers to the sequence identity or similarity between two or more individual that is result of genetic inheritance, or inheritance of the similar nucleotide sequence from a common ancestor. In certain embodiments, plants or genomes of the present disclosure may share an identity by descent as defined by a percentage of sequence identity that is derived from a common ancestor.

As used herein, the term “genetic distance” refers to the sequence similarity between the genome of two or more plants. The genetic distance between two or more plants may be defined, in certain embodiments, by the number of marker-assisted backcrosses required to recover, or essentially recover, the genome or the level of agronomic performance of one of the plants in the backcross. For example, a one marker-assisted backcross equivalent distance means that if two plants are within that distance threshold, backcrossing one of the plants to the other for a single backcross generation would be expected to bring the resulting progeny to a nearly indistinguishable level of performance to that of the backcrossed parent plant. Genetic distance may also be measured, in certain embodiments, by percent sequence identity or percent identity by descent.

As used herein, the term “plant” includes plant cells, plant protoplasts, plant cells of tissue culture from which a plant can be regenerated, plant calli, plant clumps and plant cells that are intact in plants or parts of plants. Non-limiting examples of plant parts include embryos, pollen, ovules, seeds, leaves, stems, flowers, branches, fruit, kernels, ears, cobs, husks, stalks, roots, root tips, anthers, and the like. Plants of the current disclosure include any plant species, including monocots or dicots, and may, in certain embodiments, include any crop plant, for instance forage crops, oilseed crops, grain crops, vegetable crops, fiber crops, and turf crops. In other embodiments, plant of the current disclosure may include, but are not limited to, corn (maize) (Zea mays), Brassica sp. (e.g., B. napus, B. rapa, B. juncea), alfalfa (Medicago sativa), rice (Oryza sativa), rye (Secale cereale), sorghum (Sorghum bicolor, Sorghum vulgare), wheat (Triticum aestivum), soybean (Glycine max), tobacco (Nicotiana tabacum), cotton (Gossypium barbadense, Gossypium hirsutum), oats, barley, and vegetables.

As used herein, the term “population” refers to a grouping of one or more plants. In certain embodiments, a population of plants comprises at least about 10, 50 100, 250, 500, 1,000, 5,000, 10,000, 50,000, or 100,000 or more plants.

As used herein, the terms “variety” or “cultivar” refers to a group of similar plants that by their genetic pedigrees and performance can be identified from and are distinct from other varieties or cultivars within the same species.

As used herein, “elite variety” or “elite cultivar” refers to a variety that has resulted from breeding and selection for superior agronomic performance. As used herein, the term “elite line” refers to a line that results from breeding and selection for superior agronomic performance. An “elite plant” refers to a plant belonging to an elite variety or elite line. Similarly, an “elite germplasm” or elite strain of germplasm is an agronomically superior germplasm. As used herein an “elite genome” refers to the genome of an elite plant.

As used herein, the term “introgressed” or “introgression,” when used in reference to a genetic locus, or trait conferred by a genetic locus, refers to a genetic locus or trait that has been introduced into a new genetic background, such as through backcrossing. As used herein, “trait introgression” refers to the introgression of a genetic locus that confers a trait. Introgression of a genetic locus or trait can be achieved through plant breeding methods, such as those of the present disclosure, and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the term “classic trait introgression” refers to the traditional method of introgression of a trait of interest or a locus conferring a trait of interest from the genome of a donor plant into the genome of a recipient plant. Classic trait introgression traditionally relies on repeated backcrosses of the donor parent carrying the desired trait to a recurrent parent plant, for instance containing an elite or commercial genome. The final goal of the repeated backcrossing is to achieve a plant containing both the desired trait from the donor parent plant and a high recovery of the recurrent parent plant's genome to ensure performance recovery of the elite recurrent parent plant.

As used herein, the term “donor parent” refers to a plant that contains a trait of interest or locus conferring a trait of interest in its genome for introgression into a recipient plant. The donor parent may be a homozygous (inbred), or a heterozygous (hybrid) plant.

As used herein, the term “recipient parent” refers to a plant into which a trait of interest or a locus conferring a trait of interest will be introgressed. In certain embodiments, a recipient plant may be an elite cultivar or comprise an elite genome.

As used herein, the term “recurrent parent” refers to a plant into which a trait of interest or a locus conferring a trait of interest will be introgressed and which is used for at least one backcross during a trait introgression method. In certain embodiments, a recurrent plant may be an elite cultivar or comprise an elite genome. In some embodiments, a recurrent parent is a homozygous (inbred) plant.

As used herein, the term “backcrossing” refers to a process in which a breeder repeatedly crosses progeny, for instance hybrid progeny, such as a first generation hybrid (F1), back to one of the parents of the hybrid progeny. Backcrossing can be used to introduce one or more loci, traits, or transgenes of interest from one genetic background into another and/or to recover the genome or agronomic performance or phenotype of one of the parents of the hybrid progeny.

As used herein, the term “crossing” refers to the mating of two parent plants.

As used herein, the term “marker-assisted breeding” or “marker-assisted selection” refers to a breeding or selection process where a trait or phenotype of interest is selected based on a marker, such as a genetic marker, linked to a trait or phenotype of interest, rather than selection of the trait or phenotype itself.

As used herein, the term “marker-assisted backcross” refers to a method of breeding where a trait or phenotype of interest is selected based on a marker, such as a genetic marker, linked to a trait or phenotype of interest, where the selected plant is backcrossed to one of its parent plants.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

### EXAMPLES

The following examples are included to more fully describe the invention. It should be appreciated by those of skill in the art that many modifications can be made in the specific examples which are disclosed and still obtain a similar result. Any such modifications apparent to those skilled in the art are deemed to be within the scope of the invention.

### Example 1

### Trait Integration Using Traited Parent Line

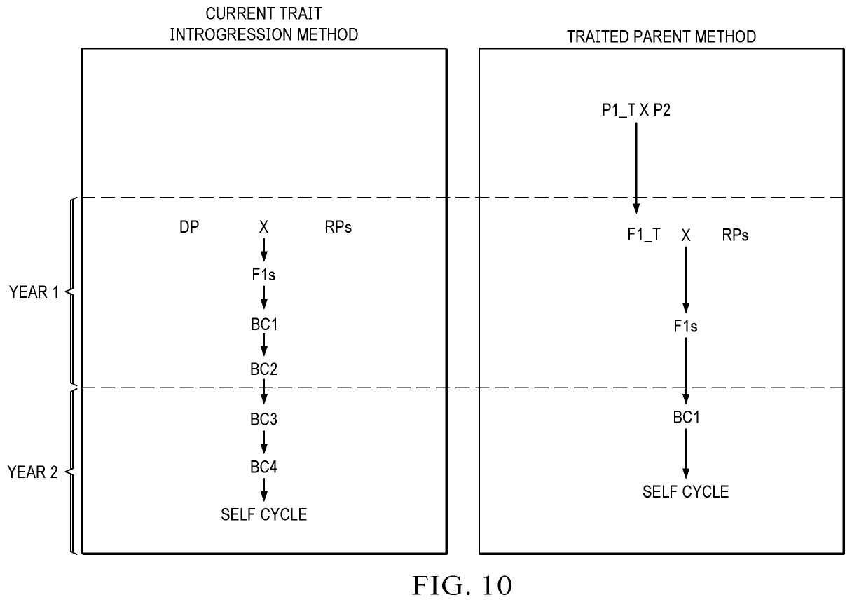

Canola trait introgression typically requires three to four backcross generations to integration a desired trait into a canola line. The typical trait integration is further slowed, as introgression must wait until a breeding decision is made to pick a trait donor line, without which the trait introgression process cannot begin (FIG. 10).

It is frequently observed that sister lines have high similarity to each other. However, as it is difficult to find a sister line as trait donor line, this example investigates the creation of traited F1 progeny by crossing traited parental lines. Thus, a novel trait introgression method for shortening the trait integration process is tested in this example.

The cross between a recipient parent and traited F1 was used to simulate the process of recipient parent crossing with their sister lines. Marker profiling is able to identify the progeny with high similarity to the recipient parent.

Canola inbred lines were derived from a cross between a first parental canola line (P1) and a second parental canola line (P2). The resulting canola inbred lines plants were used as recipient parent (RP) canola inbred lines for the new trait introgression process.

A version of the P1 canola line comprising a trait of interest (traited P1 or P1_T) was developed using traditional trait integration methods. The traited P1 was crossed with P2 to produce F1 offspring comprising the trait of interest (traited F1 or F1_T). The traited F1 canola plants were used as donor plants in the new trait integration process.

The traited F1 plants were crossed with RP canola inbred lines derived from P1 and P2, as shown in FIG. 10. The resulting offspring are referred to in this examples as F1.

The F1 individual plants were genotyped with a marker set polymorphic between population parent P1 and P2. Once genotyped, if the F1 plants reached the required similarity, the F1 plants with the highest similarity to the RP plants were selfed. If the F1 plants did not demonstrate enough similarity then the F1 plants with highest similarity to the RP plants were selected and used for further backcross to the RP.

In the instance that a backcross to RP was required, the resulting progeny of the backcross (BC1) with the highest similarity to the RP were selected and selfed. If necessary, a second backcross can be performed to achieve higher similarity.

Using this method, 412 traited canola lines were converted. Of these, 320 were selfed in the BC1 generation and achieved average IBD at 93.25; 92 were selfed in F1 generation and achieved average IBD at 90.58. This confirmed that described trait introgression method can shorten the production trait converted canola lines by up to two cycles.

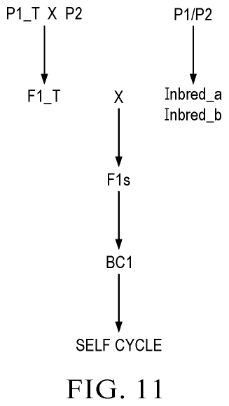

In a second experiment, shown in FIG. 11, two inbred canola lines (Inbred_a, and Inbred_b) were derived from breeding a first parental canola line (P1) and a second parental canola line (P2). A version of the P1 canola line comprising a desired herbicide trait (traited P1 or P1_T) was developed using traditional trait integration methods. The traited P1 was crossed with P2 to produce F1 offspring comprising the herbicide trait (traited F1 or F1_T). The traited F1 of the cross between P1_T and P2 was used as trait donor in the trait introgression method and was crossed with the inbred canola lines (Inbred_a and Inbred_b). A backcross to the inbred lines (BC1) followed by a selfing generation was resulted in the trait converted lines (BC1F3). The resulting BC1F3 plants were used for Infinium™ fingerprint analysis to confirm conversion quality. The trait converted canola lines reached 98.33% and 96.92% identity by descent similarity to their recurrent parents (Inbred_a and Inbred_b), respectively.

### Example 2

### Developing New Cultivars Using Reverse Selection

This example describes an example of developing of new cultivars using reverse selection.

Initially, new elite inbred lines are developed through bi-parental crosses between non-traited parental inbreds (i.e., P1 and P2). Over the course of a multi-year breeding program, elite line candidates are identified (FIG. 1, referred to therein as Line1). During the process of producing new elite lines, traited versions of the parental lines (P1 and P2) are created, using traditional backcross-based trait introgression methods or the reverse selection methods described here (FIG. 2).

A haplotype-based identity-by-descent (IBD) method, which infers the amount of genomic sharing between conversion and base germplasm, is developed. Field-based traited-testing is carried out to establish the threshold of the percentage recipient plant IBD recovery for base germplasm performance reproducibility, using existing backcross converted lines and their matching base-germplasm as training set (FIG. 3).

Forward genetic simulation is then conducted to generate 10⁶˜10⁷ virtual recombinants of the P1 and P2 genomes, as an in-silico representation of the F1-derived Double Haploid (DH) inbreds (FIG. 4). For each of the virtual recombinants, the IBD method described above is applied and the proportion of haplotype sharing between the virtual recombinant and the elite line (Line1) is estimated. The empirical distribution of the resulting IBD estimates are compared to the reproducibility threshold established during the field-based traited-testing and the empirical probability of reaching an IBD value equal-to-or-greater than the established threshold is estimated (FIG. 5).

Based on the probability of reaching an IBD value equal-to-or-greater than the established threshold estimated, and desired reliability level, the adequate DH population size is determined to reliably exceed the threshold established during the field-based traited-testing (FIG. 6). The population size is estimated using the formula z=(1−(1−x)^{y}), where x is the probability of reaching or exceeding an IBD target value, y is the population size, and z is the desired reliability. For example, when the probability of exceeding the IBD target is p=0.001, a pool of 3000 of F1-derived double haploid inbreds is required to achieve 95% reliability, as 0.95=(1−(1−0.001)³⁰⁰⁰).

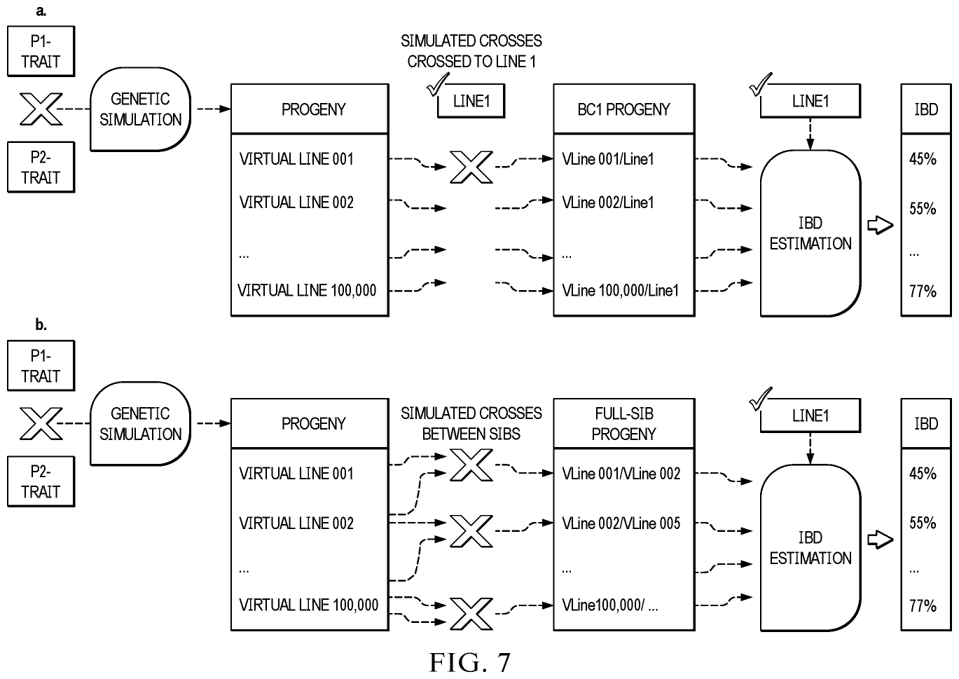

However, where there is a large genetic distance between P1 and P2 a two-step recipient plant genome IBD recovery can be employed. For instance, in such a case, the population size estimated using the formula above may exceed practical feasibility, as a high parental distance will reduce p (in the case of p=0.0005, a population size of 6000 would be required to each the same 95% reliability level). In these cases, the genetic simulation can be modified to determine the DH population size required to reach an equivalent of one or more backcrosses lower than the established IBD threshold, instead of exceeding the threshold directly from within the F1-derived DH pool (FIG. 7, panel a). The precise relationship between the number of backcrosses and the IBD metric is also in turn established by genetic simulation.

Alternatively, instead of making backcrosses to reach the IBD threshold, it is possible to make full-sib crosses within F1-derived DH pool, by choosing pairs of individuals from which high probability exists for recovering high IBD to the target Line1 (FIG. 7, panel b). The probability is in turn determined by the genetic simulation described above.

Once a population size is estimated, and it is established if backcrosses or full-sib crosses will be necessary, a final inbred may be generated. For instance, in the case of direct DH recovery, requiring no further backcrosses or full-sib crosses, the final inbred can be generated from the produced pool of F1 plants through doubling and increase. In the case of BC1 recovery, the final inbred can be generated from the produced pool of F1 plants through doubling, backcross, and increase. Where a full-sib cross is to be made, the final inbred can be generated from the produced pool of F1 plants through doubling, full-sib cross, and increase. It is noted that there are additional Mendelian segregations of the trait-of-interest when further backcrosses are made and additional genotypic- or event-based screen should to be applied to identify trait-positive F1BC1 progeny. Each inbred generated is compared to the elite target (Line1) using the IBD estimation method described above and the inbred plants exceeding the desired reproducibility IBD threshold are advanced in the breeding pipeline as Line1-with-Trait.

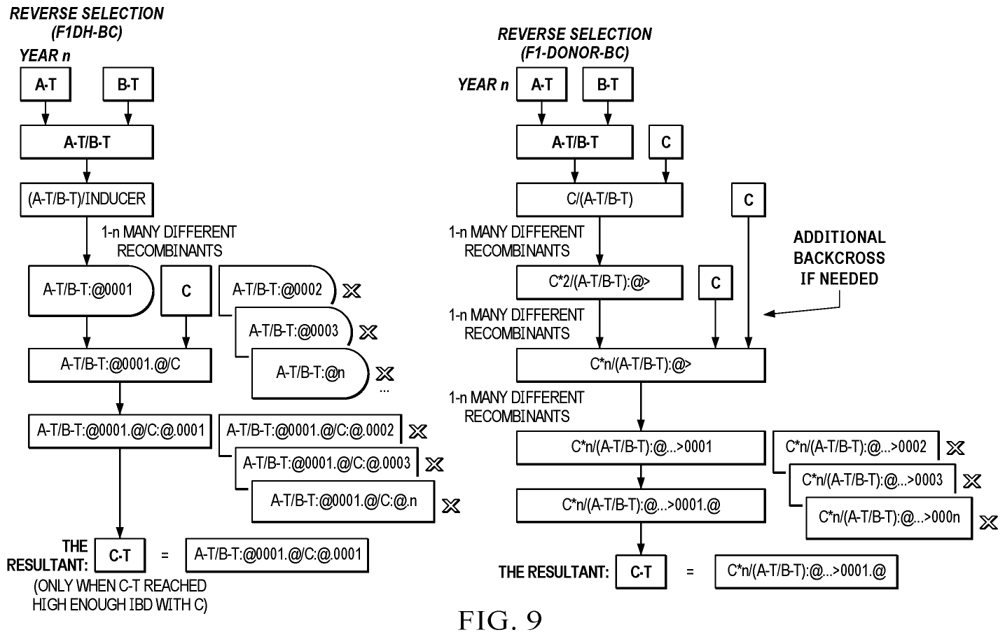

The same general approach described above can be easily customized to suit differ delivery time line and budgetary targets. This can be achieved by employing different breeding methods, such as replacing doubling with selfing or adjusting the design parameters to attain a target number of generations for the final product. Exemplary variants of the presently disclosed methods are disclosed in FIG. 9.

### Example 3

### Proof of Concept of Reverse Selection

A proof of concept experiment was conducted to evaluate the ability to develop new cultivars using the reverse selection method and the method's ability to reduce the number of cycles required to develop new cultivars.

In-silicon simulation demonstrated that the population size required to reach sufficient target genomic recovery is negatively correlated with the genetic distance between the parents as expected. For example, with a population size of 1,000 haploid corn kernels, it was estimated that 50% of current breeding pipeline for North American corn inbreds can be regenerated directly via F1-derived-DH design, with a probability of success over 95%. It was also estimated that adding an additional backcross will increase this coverage to 75% of breeding pipeline for North American corn inbreds.

A field experiment validated the in-silicon prediction within 5% margin of error. The field experiment was conducted with 15 different origins and the resultant haploid kernels were genotyped on a genotyping-by-sequence (GBS) platform. The target genome recovery, as percent identity by descent (IBD), was then estimated from the GBS genotypic data.

This proof of concept experiment demonstrated the success of the reverse selection methods to produce new cultivars. Specifically, all 15 corn origins either had kernels that already reached the desired level of target genome recovery or were within 1 backcross of such a target recovery. Depending on the target recovery level (in IBD), the kernels were divided into 2 groups: F1-derived-DH design and F1-derived-DH+BC design, and will enter the doubling phase. The two paths demonstrated the ability of the presently disclosed reverse selection methods to produce new cultivars 2-3 cycles faster than conventional trait introgression methods.

### Example 4

### Generating Non-Traited Lines Using Reverse Selection

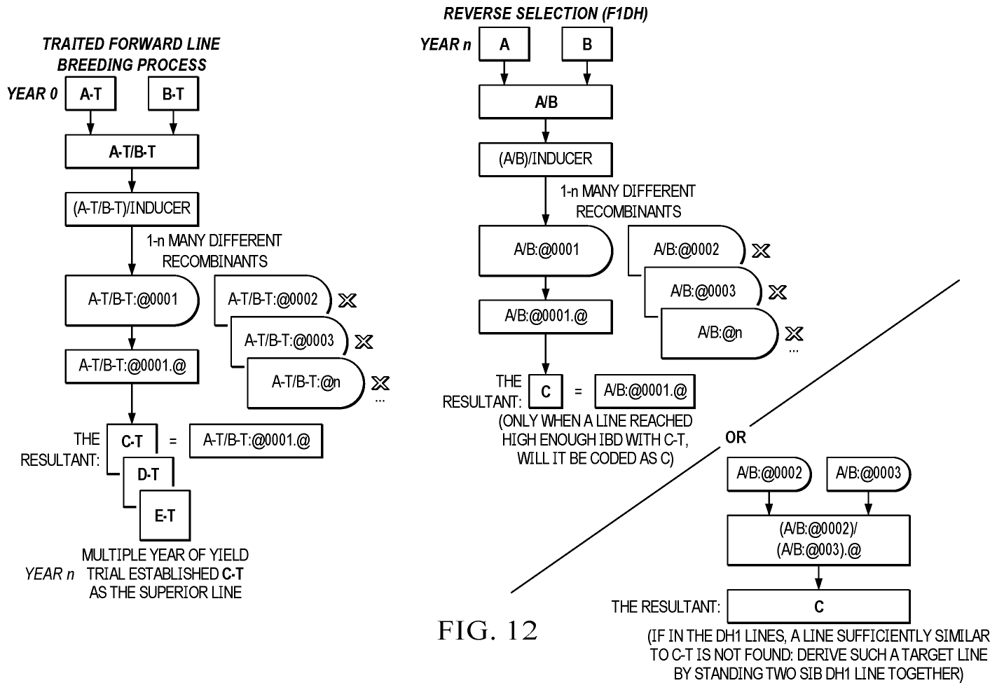

As shown in FIG. 12, generating non-traited lines via reverse selection is similar to generating traited lines, only in the former case the non-traited state is favored and in the latter case the traited state is favored. However, not all reverse selection variants are suited for the process of generating non-traited lines using reverse selection. When using reverse selection to generate a non-traited version of a traited target line, one might not have the option of backcrossing to the traited target line in order to increase its similarity, as under some regulation frameworks doing so would render the backcrossed resultant a GMO line even though it doesn't carry GMO trait. As a result, the non traited version must be generated without backcrossing, as shown in FIG. 12, resultant C is a non-traited line corresponding to traited target line C-T.

In another embodiment, and also with reference to FIG. 12: if the most similar line in the resultant progeny pool is not sufficiently similar to corresponding traited line C-T, a subsequent sibling mating step maybe included in the reverse selection process. If the sibling mating step is desired, a second, non-traited sib line from the same resultant progeny pool needs to be identified to satisfy the following condition: while each single one line of the sib pair is not sufficiently similar to the target line C-T, they are complementary to each other and when combined can produce a progeny of sufficient similarity to C-T. Where one member of the said sib pair might have a chromosome region different from C-T, the selected complementary member of the said pair must provide the required chromosome region matching C-T. The two non-traited resultants may then be mated to achieve a non-traited resultant line with sufficient similarity to corresponding traited line C-T by the recombination of those complementary regions.

What is claimed is:

1. A method of generating a traited version of a target line, the method comprising:
a) providing a first parent and a second parent, wherein at least one of the first parent and the second parent carries at least one trait of interest;
b) estimating a traited progeny population size based at least on genetic information associated with each of the first parent, the second parent, and the target line;
c) generating a traited progeny population by crossing the first parent with the second parent, wherein a size of the traited progeny population is equal to or greater than the traited progeny population size; and
d) selecting, from the traited progeny population, at least one traited target line based on a genetic similarity between the traited target line and the target line.

2. The method of claim 1, wherein estimating a traited progeny population size comprises:
a) generating a set of virtual genomes by simulating recombinations of the genomes of the first parent and the second parent;
b) estimating a similarity between each member of the set of virtual genomes and a genome of the target line;
c) comparing each of the estimated similarities to a similarity threshold;
d) determining a proportion of the set of virtual genomes whose estimated similarities to the genome of the target line is equal to or exceeds the similarity threshold; and
e) estimating, based on the proportion, a probability of a recombination between the first parent and the second parent of generating a line whose similarity to the target line is equal to or exceeds the similarity threshold.

3. The method of claim 2, wherein estimating a similarity between each member of the set of virtual genomes and the genome of the target line is based on an identity-by-descent method.

4. The method of claim 3, wherein the identity-by-descent method is a haploid-based identity-by-descent method.

5. The method of claim 1, further comprising crossing the at least one traited target line with the target line.

6. The method of claim 1, wherein the target line is an inbred corn line.

7. The method of claim 1, wherein the target line is an inbred rapeseed line.

8. The method of claim 1, wherein the at least one trait of interest comprises at least one agronomic trait of interest.

9. The method of claim 8, wherein the at least one agronomic trait of interest is associated with any combination of herbicide tolerance, insect control, increased plant pathogen resistance, enhanced oil composition, increased water use efficiency, increased yield, increased drought resistance, increased seed quality, improved nutritional quality, increased nitrogen use efficiency, or tolerance to nitrogen stress.

10. A system for generating a traited version of a target line, the system comprising:
a) a breeding pipeline associated with a target environmental region;
b) a first parent and a second parent, wherein at least one of the first parent and the second parent carries at least one trait of interest;
c) a computing device in communication with a data structure and a memory and configured to estimate a traited progeny population size based at least on genetic information associated with each of the first parent, the second parent, and the target line;
d) a means of generating a traited progeny population by crossing the first parent with the second parent, wherein a size of the traited progeny population is equal to or greater than the traited progeny population size; and
e) a means of selecting, from the traited progeny population, at least one traited target line based on a genetic similarity between the traited target line and the target line;
wherein a plant derived from the at least one traited target line is planted in a growing space and directed into the breeding pipeline.

11. The system of claim 10, wherein estimating a traited progeny population size comprises:
a) generating a set of virtual genomes by simulating recombinations of the genomes of the first parent and the second parent;
b) estimating a similarity between each member of the set of virtual genomes and a genome of the target line;
c) comparing each of the estimated similarities to a similarity threshold;
d) determining a proportion of the set of virtual genomes whose estimated similarities to the genome of the target line is equal to or exceeds the similarity threshold; and
e) estimating, based on the proportion, a probability of a recombination between the first parent and the second parent of generating a line whose similarity to the target line is equal to or exceeds the similarity threshold.

12. The system of claim 11, wherein estimating a similarity between each member of the set of virtual genomes and the genome of the target line is based on an identity-by-descent method.

13. The system of claim 12, wherein the identity-by-descent method is a haploid-based identity-by-descent method.

14. The system of claim 10, further comprising crossing the at least one traited target line with the target line.

15. The system of claim 10, wherein the target line is an inbred corn line.

16. The system of claim 10, wherein the target line is an inbred rapeseed line.

17. The system of claim 10, wherein the at least one trait of interest comprises at least one agronomic trait of interest.

18. The system of claim 17, wherein the at least one agronomic trait of interest is associated with any combination of herbicide tolerance, insect control, increased plant pathogen resistance, enhanced oil composition, increased water use efficiency, increased yield, increased drought resistance, increased seed quality, improved nutritional quality, increased nitrogen use efficiency, or tolerance to nitrogen stress.

19. A method of generating a genetically modified version of a target line, the method comprising:
a) providing a first parent and a second parent, wherein at least one of the first parent and the second parent carries a genetic modification of interest;
b) estimating a genetically modified progeny population size based at least on genetic information associated with each of the first parent, the second parent, and the target line;
c) generating a genetically modified progeny population by crossing the first parent with the second parent, wherein a size of the genetically modified progeny population is equal to or greater than the genetically modified progeny population size; and
d) selecting, from the genetically modified progeny population, at least one genetically modified target line based on a genetic similarity between the genetically modified target line and the target line.

20. The method of claim 19, further comprising crossing the at least one genetically modified target line with the target line.
